(19) United States
(12) Patent Application Publication
(10) Pub. No.: US 2005/0177133 A1
(43) Pub. Date: Aug. 11, 2005

(54) External urinary catheter

(76) Inventors: Henrik Lindenskov Nielsen, Smorum (DK); Soren Kirkegard Nielsen, Charlottenlund (DK)

Correspondence Address: JACOBSON HOLMAN PLLC, 400 SEVENTH STREET N.W., SUITE 600, WASHINGTON, DC, 20004, US

(21) Appl. No.: 10/520,817

(22) PCT Filed: Jul. 3, 2003
(86) PCT No.: PCT/DK03/00470

(30) Foreign Application Priority Data
Jul. 8, 2002 (DK) PA200201070

(51) Int. Cl. A61M 27/00

(52) U.S. Cl. 604/544

(57) ABSTRACT

The external urinary catheter for use as an external catheter comprises a tip portion (3) and a sheath portion (1). The external urinary catheter is manufactured by thermoplastic processing of a material comprising at least one transparent and/or thermoplastic elastomer and at least one plasticizer.

### FIELD OF THE INVENTION

The present invention relates to an external urinary catheter, comprising a tip portion and a sheath portion, said external urinary catheter being manufactured by thermoplastic processing. The invention further relates to a kit comprising such an external urinary catheter and a method of manufacture of the external urinary catheter.

### BACKGROUND OF THE INVENTION

External urinary catheters are conventionally used in urinary catheter devices for aiding male urinary incontinence and for use in hospitals in connection with treatment and surgery of urethral disorders. Such an external urinary catheter normally comprises a sheath or body portion enclosing the shaft of the penis, and a tip portion that is provided with a comparatively short discharge tube which via a hose is connected to a urine collection bag that is eg. fastened to the bed or the leg of the user. Alternatively, the external urinary catheter, or condom catheter, is arranged externally on the penis and may be a corona urisheath sealing against the foreskin.

Traditionally, such external urinary catheters have been produced like other sheath-shaped elements to be arranged on parts of the human body, ie. of latex in a multi-step dipping process, during which a mandrel of the same size and shape as the part of the body concerned is dipped repeatedly into a latex solution, which in between immersions is cured on the mandrel.

This solution, however, suffers from several deficiencies. Firstly, latex is a natural product, which may vary in respect of quality and composition and may ia. contain allergy-inducing substances. Secondly, the storage conditions are essential to the lifetime of latex products, as the lifetime may be shortened by the fact that their structure contains double linkings that are sensitive to UV radiation, resulting in a decomposition of the material, whereby the properties of the product may deteriorate drastically. Thirdly, the latex dipping process is a relatively time-consuming process, and furthermore, ammonia is normally employed and often also methanol and inorganic salts, which entails that a continuous destruction of chemical residues that are detrimental to the environment must take place.

To avoid or at least reduce these disadvantages a number or propositions have been made relating to the production of external urinary catheters and similar products using different materials and methods.

Applicant's international published application No. WO 91/17728 discloses an external urinary catheter and a method for its manufacture, in which the external urinary catheter is manufactured by thermoplastic processing of a non-preprocessed compound thermoplastic material. The elasticity of the material is increased by the addition of a paraffinic process oil. More specifically, the discharge tube at the end of the tip is produced by injection moulding in a tool, whereas the thin-walled body portion is produced integrally with the discharge tube by a subsequent controlled extrusion and blow moulding.

This provides for a number of process-related advantages in combination with a significantly enhanced freedom of choice in respect of product design, among other things as concerns variations of the wall thickness in the longitudinal direction of the product.

However, although the material used has excellent properties as regards ia. the processibility in the thermoplastic process, it has a low water vapour permeability. In combination with the fact that the external urinary catheter is usually worn for an extended period of time, this entails that the environment under the external urinary catheter becomes more or less constantly humid. In turn, this entails that the skin under the external urinary catheter is prone to be irritated, and in severe cases, maceration may occur. This problem is underlined by the fact that the external urinary catheter in its condition of use is opaque, ia. because it is necessary to apply a talcum powder to the external urinary catheter in order to prevent that the material sticks to itself during manufacture, storage and use, in particular to secure that successive windings in the rolled-up body portion is prevented from sticking together in the delivery condition of the external urinary catheter. The talcum makes it impossible to inspect the area enclosed by the external urinary catheter without removing the external urinary catheter.

In order to overcome at least the disadvantages of possible allergy induced by natural products such as latex and the lack of possibility of inspecting the area due to the opaqueness of at least the sheath portion, use of silicone as a base material for producing the external urinary catheter has been suggested. However, although silicone has a higher permeability than most materials used in external catheters, it is still not as breathable as desired. Consequently, a silicone catheter suffers from the same disadvantage, although to a less extent, as the external urinary catheter disclosed in the above-mentioned WO 91/17728.

WO 96/29962 and WO 96/29963 both address these problems and each discloses a condom catheter manufactured by dipping of mandrel in a biocompatible polyurethane. In the first-mentioned document, a conical portion forming the tip portion of the condom catheter is supplied as a separate prefabricated part of a resilient material such as PVC or polyurethane, and the sheath portion is formed during the dipping process, thus integrally connecting the sheath portion with the conical portion. In the last-mentioned document, the conical portion and the sheath portion are both formed by dipping; however in two different polyurethane formulations.

As described in the above, such a dipping process is disadvantageous in that the production time is necessarily extensive.

### SUMMARY OF THE INVENTION

With this background it is an object of the present invention to provide an external urinary catheter, which provides for an improved comfort for the user, and in which at least some of the above-mentioned disadvantages of the prior art devices are remedied, in particular with respect to the manufacturing conditions.

In a first aspect of the invention, this and further objects are met by the provision of an external urinary catheter of the kind mentioned in the introduction, which is characterized in that, in a position of use, at least one area of the external urinary catheter is transparent and/or permeable.

In this manner, an external urinary catheter has been provided that has the desired properties with respect to user comfort. In case at least one area of the external urinary catheter is transparent in its condition of use, it is possible at all times to inspect the corresponding area of the penis and, if necessary, remove the external urinary catheter. In case the external urinary catheter is permeable, the skin beneath is able to breathe. Furthermore, the external urinary catheter may be manufactured in a thermoplastic process with the advantages associated with such processes, such that a substantially clear external urinary catheter is provided, which may furthermore be produced in a simple and cost-effective manner. At least a part of the urisheath is sufficiently clear to allow inspection of an area of the skin beneath the urisheath with the urisheath material in contact with the skin.

In an advantageous embodiment, the external urinary catheter is made from a material comprising at least one transparent thermoplastic elastomer and at least one plasticizer.

As commercially available transparent thermoplastic elastomers are normally not sufficiently flexible to be used in external urinary catheters, the addition of plasticizers is utilized in order to provide the external urinary catheter with the desired degree of flexibility and elasticity. It has been believed that the elastic memory and/or the processibility of the thermoplastic incorporating the plasticizer would not be sufficient in order to manufacture such an external urinary catheter by thermoplastic processing. However, it turns out that it is possible to obtain a material having properties that are satisfactory in order to fulfil the above-mentioned demands on the external urinary catheter, even with the addition of a relatively large amount of the plasticizer.

In a further development of this embodiment, said material comprises a polystyrene polyethylene/butylene polystyrene compound, another styrenic elastomer compound or elastomeric metalocen polyethylene or metalocen polypropylene, and at least one slip additive.

The slip additive may be an amide, such as erucamide, stereamide or oleamide or any other suitable slip additive.

The degree of transparency is preferably chosen such that said at least one area has a haze value (according to ASTM Standard Test Method D1003-61) lower than 30%, preferably lower than 15%.

In another advantageous embodiment, the external urinary catheter is made from a material comprising at least one permeable thermoplastic elastomer and at least one plasticizer.

As commercially available permeable thermoplastic elastomers are normally not sufficiently flexible to be used in external urinary catheters, the addition of plasticizers is utilized in order to provide the external urinary catheter with the desired degree of flexibility and elasticity. It has been believed that the addition of plasticizers to thermoplastic elastomers would deteriorate the permeability of the material, and that the elastic memory and/or the processibility of the thermoplastic incorporating the plasticizer would not be sufficient in order to manufacture such an external urinary catheter in by thermoplastic processing. However, it turns out that it is possible to obtain a material having properties that are satisfactory in order to fulfil the above-mentioned demands on the external urinary catheter, even with the addition of a relatively large amount of the plasticizer.

In a further development of this embodiment, the material has such a degree of breathability that a film of said material having a thickness of approximately 0.2 mm has a permeability of at least 500 g/m² pr. 24 hours, preferably at least 1000 g/m² pr. 24 hours, more preferably at least 1500 g/m² pr. 24 hours.

The thermoplastic elastomer may be a polyamide-polyether block copolymer, a polyether ester, a thermoplastic urethane or any other suitable material, as long as they fulfil the demands with respect to the desired permeability, transparency and/or processibility.

The plasticizer may be a citrate plasticizer, a sulphone amide, a benzoate ester or any other suitable material, which is compatible with the thermoplastic elastomer.

In order to increase the melt strength, i.e. the strength of the material in a fluid state, the material may further comprise an addition of a polymer.

Such a polymer may be a polyethylene elastomer, a polyethylene-vinyl acetate copolymer, a graft polyolefin maleic anhydride, an amorphous ethylenepropylene-diene terpolymer, an ionomer or any other suitable material.

In an embodiment of the external urinary catheter, the inner side of the sheath portion is provided with an integral layer of a pressure-sensitive adhesive and the outer side of the sheath portion with an adhesive-rejecting layer.

However, the attachment of the external urinary catheter may, in a second aspect of the invention, be provided by means of a kit comprising an external urinary catheter and a separate adhesive element, or, alternatively, by a kit comprising an external urinary catheter and interlocking elements.

In a third aspect of the invention, a method of manufacturing an external urinary catheter comprising a tip portion and a sheath portion by subjecting a base material to one or more steps in a thermoplastic process is provided, said method being characterized in that said base material comprises at least one transparent and/or permeable thermoplastic elastomer and at least one plasticizer.

The tip portion may be produced by injection moulding and said sheath portion by extrusion, extrusion blow moulding, injection blow moulding or cold rolling.

The tip portion may form an integral part with the sheath portion or may be produced as a separate unit which is subsequently connected with the sheath portion.

Likewise, it is possible to produce the external urinary catheter entirely by extrusion blow moulding.

Further features and advantages may readily be appreciated from the following detailed description.

### BRIEF DESCRIPTION OF THE DRAWINGS

In the following the invention will be described in further detail with reference to the schematic drawings, in which

FIG. 1 shows a sectional side view of an embodiment of the external urinary catheter according to the invention; and

FIG. 2 shows a sectional side view of another embodiment of the external urinary catheter according to the invention.

### DETAILED DESCRIPTION OF PREFERRED EMBODIMENTS

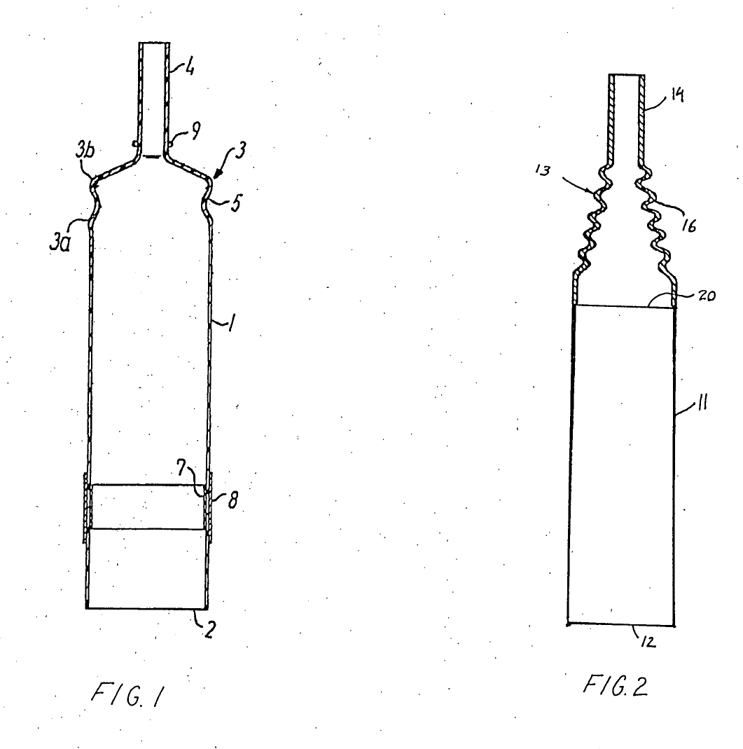

The configuration of the external urinary catheter shown in FIGS. 1 and 2 corresponds in substance to Applicant's above-mentioned published international application WO 91/17728. In the embodiment in FIG. 1, the external urinary catheter comprises a soft thin-walled and flexible sheath portion 1 with an open end 2. At the opposite end the sheath portion 1 contracts towards a tip or end portion 3. The tip portion 3 comprises an anti-kinking chamber provided by first and second sections 3a and 3b, and a constriction 5. The constriction 5 furthermore accommodates the sheath portion in the rolled-up delivery condition. The tip portion 3 furthermore comprises a discharge or drainage tube 4, which by means of a hose may be connected to a urine collection bag (not shown) that is normally fastened to the user's leg. In the embodiment shown in FIG. 1, the external urinary catheter is provided with a circumferential bead 9.

In order to secure the external urinary catheter to penis of the user, part of the inner side of the sheath portion may in a manner known per se be provided with an integral layer 7 of a pressure-sensitive adhesive, and on the outer side with a matching adhesive-rejecting layer 8 in order to prevent successive windings in the rolled-up body portion 6 from sticking together. The adhesive, which may have a relatively high permeability to water vapour may also be supplied as a separate element that is to be connected with the external catheter. The attachment of the external catheter may be performed in other ways, e.g. by the use of interlocking elements such as Velcro®.

The details in the configuration of the external urinary catheter are not decisive. Thus, the external urinary catheter may have any other shape, eg. as shown in FIG. 2, in which a tip portion 13 comprises a corrugated section 16 that functions as anti-kinking chamber. The corrugated section 16 allows a significant angular displacement between the discharge tube 14 and the sheath portion 11 and is furthermore able to absorb tensile and pressure load on the external urinary catheter. As is suggested by line 20, the tip portion 13 may be produced in one process and the sheath portion 11 in another and subsequently be joined to each other in any suitable manner.

As is the case with the external urinary catheter disclosed in the above-mentioned WO 91/17728, the external urinary catheter according to the invention is produced by thermoplastic processing. However, as a base material a compound of a thermoplastic elastomer and a plasticizer other than the paraffinic process oil used in the process disclosed in the above-mentioned WO document is used.

In case the external catheter is desired to be transparent but not necessarily breathable, the material used may comprise a polystyrene polyethylene/butylene polystyrene compound, another styrenic elastomer compound or elastomeric metalocen polyethylene or metalocen polypropylene, and at least one slip additive, preferably a slip additive not containing talcum. The elastomeric metalocen polyethylene should have a controlled co-monomer content and the elastomeric polypropylene should have controlled tactic and atactic domains. The slip additive may be an amide, such as erucamide, stereamide or oleamide or any other suitable slip additive.

The term “transparent” should in the present context be interpreted as indicating such a degree of transparency that it is possible to inspect the area of the penis that corresponds to the at least one area possessing the transparent property. The area may e.g. have a haze value (according to ASTM Standard Test Method D1003-61 “Standard Test Method for Haze Luminous Transmittance of Transparent Plastics” using a BYK Gardner Hazegard Plus) lower than 30% and preferably lower than 15% measured on the actual material thickness of the external urinary catheter.

A suitable thermoplastic elastomer is chosen in consideration of a number of factors, among others transparency, permeability and processibility. Polyamide-polyether block copolymers, or polyether block amides, polyether esters and thermoplastic urethanes may be utilized as well as chlorinated elastomers.

In case the external catheter is desired to be breathable but not necessarily possess a high degree of transparency, other components are conceivable.

The plasticizer may be a citrate plasticizer, a sulphone amide, a benzoate ester or any other suitable material. Evidently, the plasticizer should be compatible with the elastomer and be able to provide the material with the desired properties as regards deformation ability, flexibility and elasticity, without reducing the permeability and processibility of the material significantly.

Furthermore, the plasticizer should have a relatively high boiling point, and the elastomer a relatively low melting point. The melting point of the elastomer should be lower than the boiling point of the plasticizer such that both are fluid within the range of suitable process temperatures. A material that comprises 10-60% citrate plasticizer, eg. Citrofol® BII (acetyltributyl citrate) or Citrofol® BI (tributyl citrate) from Jungbunzlauer, and 40-90% polyamide-polyether block copolymer, eg. Pebax® 2533 from Atofina, has proven particularly advantageous. Other suitable examples are Citroflex® A-6 (acetyltri-n-hexyl citrate) and Citroflex® B-6 (butyryltri-n-hexyl citrate). All percentages are expressed in percentage by weight.

Common demands on the elastomer and the plasticizer are that they should be biocompatible and toxicologically harmless.

In the case of a breathable external catheter, the material should possess such a degree of permeability, or breathability, that the user experiences an increased comfort even during prolonged use of the external urinary catheter, as the skin under the external urinary catheter in its position of use is able to “breathe”. This makes it possible to use the external catheter for relatively long periods of time, instead of having to remove the external catheter in order to relieve the skin by means of intermediate periods, in which e.g. a urinal or an absorbent article. It is noted that the term “permeable” should be interpreted as meaning permeable to water vapour and similar gases. Suitable permeability properties are such that a film of said material having a thickness of approximately 0.2 mm has a permeability of at least 500 g/m² pr. 24 hours, preferably at least 1000 g/m² pr. 24 hours, more preferably at least 1500 g/m² pr. 24 hours.

The elastomer and the plasticizer, and possibly additional substances as will be described further on, are, in a manner known per se, processed in a batch or continuous compounding into a base material that is subjected to one or more steps in a thermoplastic process.

It should furthermore be mentioned that the external urinary catheter may be manufactured from a thermoplastic elastomer that is permeable as well as transparent. This entails an optimum user comfort, as in addition to the breathable feature, it is possible to inspect at least the area of penis corresponding to the transparent area of the external urinary catheter in its condition of use.

The thermoplastic process may be carried out as described in detail in Applicant's above-mentioned WO 91/17728, in which at least the discharge tube of the tip portion is produced by injection moulding and the sheath portion by a subsequent controlled pull extrusion and blow moulding process.

Furthermore, the principle disclosed in DK patent publication No. 150792 may be used.

Alternatively, the sheath portion may be provided by means of other processes, e.g. injection blow moulding or cold rolling.

As described in connection with the embodiment of FIG. 2, the tip portion may be produced as a separate unit in one process which is subsequently connected with the sheath portion which is produced in another process.

It is further conceivable to manufacture the external urinary catheter in a single step, eg. entirely by extrusion blow moulding.

As mentioned in the above, the amount of the plasticizer used in the base material is chosen such that the external urinary catheter is ia. sufficiently soft, an amount of approximately 50% leading to an optimum softness. However, in case the amount of plasticizer is relatively high, the melt strength, i.e. the strength of the material in the fluid state is reduced, whereby processibility is affected, and the breaking strength and the elastic memory of the finished external urinary catheter are reduced. In order to counteract these effects, the material may further comprise an addition of a polymer having a high viscosity. Such a polymer may be chosen from the group of polyethylene elastomers, polyethylene-vinyl acetate copolymers, graft polyolefin maleic anhydrides, amorphous ethylene-propylene-diene terpolymers, ionomers or any other suitable material.

A particularly advantageous material has proven to comprise 50-60%, polyamide-polyether block copolymer, eg. 55% Pebax® 2533 SA 01, 10-20% amorphous ethylenepropylene-diene terpolymer, eg. 15% Nordel® IP 4520 and 20-40% citrate plasticizer, eg. 30% Citrofol® BII.

By this particular combination, a substantially transparent external urinary catheter having satisfactory elasticity properties is provided. Tests have shown that the addition of the viscous polymer increases the melt strength of the compound significantly.

Furthermore, the external urinary catheter is substantially free of stickiness, which entails that it does not become necessary to apply talcum to the external urinary catheter in order to avoid that the material sticks to itself.

A second example of an advantageous material has proven to be 50% Pebax® 2533 SA 01, 10% Surlyn 9320 and 40% Citrofol® BII or any other suitable plasticizer like Citroflex® A-6 or B-6.

A third example of an advantageous material is 50% Pebax® 2533 SA 01, 10% Fusabond MF 416 D or Fusabond MF 493 D and 40% Citrofol® BII or any other suitable plasticizer like Citroflex® A-6 or B-6.

The choice of the elastomer and plasticizer, and the respective amounts, may be optimised in respect of any particular demands. For instance, it is of course conceivable to utilize more than one kind of elastomer and plasticizer, respectively, that have mutually different properties.

It is to be noted that the thickness of the external catheter has an influence on the permeability. Consequently, catheters having a large thickness and made from a material having a relatively high water vapour permeability may have substantially the same or better permeability properties than a thinner catheter made from a less permeable material. Thus, it is conceivable to optimise the permeability properties with a suitable combination of thickness and material.

In order to reduce any tendency of stickiness of the material, a release and/or anti-blocking agent, known per se, such as eg. an amide or an amorphous silica, may be added.

### EXAMPLE

A film was prepared on the basis of each of the following compounds:
1.58% polyether block amide, 42% citrate plasticizer
2.67% polyether block amide, 33% citrate plasticizer

The films were tested according to a test method used to measure the permeability of adhesives and films. The method is carried out by means of an apparatus comprising: Water permeation chambers with lids (hole of 20 mm in diameter), 0.9% saline solution. Analytical balance: Exsiccator and incubator 37° C. or if possible controlled-climate unit with fixed humidity level. Dial gauge, pipette, sample cutting tool, 25 mm in diameter. A double or triple test is made: Samples are cut and their thickness measured. The chambers are filled with approx. 5 ml of saline solution and the test cuts are placed over them (for adhesives, adhesive surface facing downwards), the lid rings are fitted and tightened. The chambers are then weighed (=zero value) and placed in exsiccator with dried silica gel at 37° C. or in controlled-climate unit at 37° C. and 15% humidity. The chambers are weighed after 24 hours, if necessary more than once—to be specified as required. The openings of the chambers were facing upwards. The results after 24 hours were calculated and are listed in Table 1 below together with results from two commercially available external urinary catheters, viz. 3: corresponding to WO 91/17728 and 4: a silicone external urinary catheter.

TABLE 1

| | Film thickness | Weight loss | Permeability |
|---|---|---|---|
| | [nun] | [g] | [g/m2] |
| 1 | 0.23 | 0.2191 | 2191 |
| 1 | 0.25 | 0.1945 | 1945 |
| 1 | 0.25 | 0.2082 | 2082 |
| 2 | 0.17 | 0.2618 | 2618 |
| 2 | 0.16 | 0.2673 | 2673 |
| 2 | 0.18 | 0.2633 | 2633 |
| 3 | 0.29 | 0.0058 | 58 |
| 3 | 0.25 | 0.0062 | 62 |
| 3 | 0.20 | 0.0070 | 70 |
| 4 | 0.18 | 0.1375 | 1375 |
| 4 | 0.18 | 0.1310 | 1310 |
| 4 | 0.19 | 0.1314 | 1314 |

As may be seen, an external urinary catheter according to the invention has a permeability that is larger than the permeability of the silicone external urinary catheter by a factor of almost 2.

The invention should not be regarded as being limited to the embodiment described in the above but various modifications may be carried out without departing from the scope of the following claims.

For example, although the invention has been described only with respect to external urinary catheters for positioning on the penis, it is of course possible to apply it to other products, in which large demands are made to the contact between the product and the skin. Evidently, the invention may as well be applied to contraceptive condoms, fingerstalls etc.

1-22. (canceled)

23. An external urinary catheter, comprising a tip portion and a sheath portion, said external urinary catheter being manufactured by thermoplastic processing, characterized in that, in a position of use, at least one area of the external urinary catheter is transparent and/or permeable.

24. An external urinary catheter as claimed in claim 23, in which the external urinary catheter is made from a material comprising at least one transparent thermoplastic elastomer and at least one plasticizer.

25. An external urinary catheter as claimed in claim 24, in which said material comprises a polystyrene polyethylene/butylene polystyrene compound, another styrenic elastomer compound or elastomeric metalocen polyethylene or metalocen polypropylene, and at least one slip additive.

26. An external urinary catheter as claimed in claim 25, in which said at least one slip additive is an amide, such as erucamide, stereamide or oleamide or any other suitable slip additive.

27. An external urinary catheter as claimed in claim 23, in which the said at least one area has a haze value (according to ASTM Standard Test Method D1003-61) lower than 30%, preferably lower than 15%.

28. An external urinary catheter as claimed in claim 23, in which the external urinary catheter is made from a material comprising at least one permeable thermoplastic elastomer and at least one plasticizer.

29. An external urinary catheter as claimed in claim 28, in which a film of said material having a thickness of approximately 0.2 mm has a permeability of at least 500 g/m²pr. 24 hours, preferably at least 1000 g/m² pr. 24 hours, more preferably at least 1500 g/m²pr. 24 hours.

30. An external urinary catheter as claimed in claim 28, in which the thermoplastic elastomer is a polyamide-polyether block copolymer, a polyether ester, a thermoplastic urethane or any other suitable material.

31. An external urinary catheter as claimed in claim 29, in which the thermoplastic elastomer is a polyamide-polyether block copolymer, a polyether ester, a thermoplastic urethane or any other suitable material.

32. An external urinary catheter as claimed in claim 23, in which the external urinary catheter is made from a material comprising at least one transparent and permeable thermoplastic elastomer and at least one plasticizer.

33. An external urinary catheter as claimed in claim 24, in which the plasticizer is a citrate plasticizer, a sulphone amide, a benzoate ester or any other suitable material.

34. An external urinary catheter as claimed in claim 24, in which said material comprises 10-60% citrate plasticizer and 40-90% polyamide-polyether block copolymer.

35. An external urinary catheter as claimed in claim 24, in which said material further comprises an addition of a polymer.

36. An external urinary catheter as claimed in claim 28, in which said material further comprises an addition of a polymer.

37. An external urinary catheter as claimed in claim 32, in which said material further comprises an addition of a polymer.

38. An external urinary catheter as claimed in claim 35, in which said polymer is a polyethylene elastomer, a polyethylene-vinyl acetate copolymer, a graft polyolefin maleic anhydride, an amorphous ethylene-propylene-diene terpolymer, an ionomer or any other suitable material.

39. An external urinary catheter as claimed in claim 24, in which said material comprises 50-60% polyamide-polyether block copolymer, 10-20% amorphous ethylene-propylene-diene terpolymer and 20-40% citrate plasticizer.

40. An external urinary catheter as claimed in claim 24, in which the material further comprises a release and/or anti-blocking agent, such as eg. an amide or an amorphous silica.

41. An external urinary catheter as claimed in claim 23, in which the inner side of the sheath portion is provided with an integral layer of a pressure-sensitive adhesive and the outer side of the sheath portion with an adhesive-rejecting layer.

42. A kit comprising an external urinary catheter as claimed in claim 23 and a separate adhesive element.

43. A kit comprising an external urinary catheter as claimed in claim 23 and interlocking elements.

44. A method of manufacturing an external urinary catheter comprising a tip portion and a sheath portion by subjecting a base material to one or more steps in a thermoplastic process, characterized in that said base material comprises at least one transparent and/or permeable thermoplastic elastomer and at least one plasticizer.

45. A method as claimed in claim 44, in which said tip portion is produced by injection moulding and said sheath portion by extrusion, extrusion blow moulding, injection blow moulding or cold rolling.

46. A method as claimed in claim 45, in which the tip portion is produced as a separate unit which is subsequently connected with the sheath portion.

47. A method as claimed in claim 44, in which said external urinary catheter is produced entirely by extrusion blow moulding.
